(19) United States
(12) Patent Application Publication
(10) Pub. No.: US 2023/0226238 A1
(43) Pub. Date: Jul. 20, 2023

(54) A Device for Retaining an Object in a Sterilization/Disinfection Apparatus

(71) Applicant: Saban Ventures Pty Limited, Lane Cove, NSW (AU)

(72) Inventors: Rigor Del Mundo Aspa, Sutherland, NSW (AU); Timothy Edward Montanaro, Wollstonecraft, NSW (AU); Christopher Hibbard, North Balgowlah, NSW (AU); Steven Paul Farrugia, Lugarno, NSW (AU); Say Kwang Lim, North Ryde, NSW (AU); Gianluigi Bortoluzzi, Lidcombe, NSW (AU)

(73) Assignee: Saban Ventures Pty Limited, Lane Cove, NSW (AU)

(21) Appl. No.: 18/001,293

(22) PCT Filed: Jul. 16, 2021
(86) PCT No.: PCT/AU2021/050766

§ 371 Date: Dec. 9, 2022

(30) Foreign Application Priority Data
Jul. 17, 2020 (AU) 2020902481

(51) Int. Cl. A61L 2/26; A61L 2/18; A61L 2/10; A61L 2/08; A61L 2/28; A61B 90/70

(52) U.S. Cl. CPC A61L 2/26; A61L 2/186; A61L 2/10; A61L 2/081; A61L 2/28; A61B 90/70; A61L 2202/123; A61L 2202/122; A61L 2202/11; A61L 2202/24

(57) ABSTRACT

Embodiments of the invention are directed to a device (10) for retaining an object in a disinfection apparatus. The device includes at least one support member (12) for contact engagement with one or more contact surfaces of the object. The at least one support member is formed from a material that allows a disinfection media to reach the one or more contact surfaces of the object such that the one or more contact surfaces are disinfected during a disinfection process.

### FIELD OF INVENTION

The present invention relates to a device for retaining an object in sterilization/disinfection apparatuses. In particular, the present invention relates to devices for retaining one or more medical objects in sterilization/disinfection apparatuses.

For example, systems and methods in accordance with embodiments of the invention may be used for retaining ultrasonic probes in sterilization/disinfection apparatuses. For convenience, much of this application will reference that context; however, it will be appreciated that the invention is not limited to this particular field of use.

### BACKGROUND

The following discussion of the prior art is intended to place the invention in an appropriate technical context and enable the associated advantages to be fully understood. However, any discussion of the prior art throughout the specification should not be considered as an admission that such art is widely known or forms part of the common general knowledge in the field.

Ultrasound probes are used for a variety of intra cavity procedures including intra rectal, intra vaginal and oesophageal examination as well as surface use. These probes are typically constructed integrally with the power and data cord used to link the probe with a control console.

To avoid cross-contamination, these probes should be disinfected or sterilized. For this reason, a sterilization/disinfection apparatus having a hollow chamber is typically employed. When such an apparatus is used, the probe is typically retained in position inside the chamber by using a resilient clamp to engage the probe's cord thereby allowing the probe to be suspended in position within the cavity. Once the probe is in position, the door of the sterilization/disinfection apparatus can be closed thereby sealing the chamber.

The sterilization process would now begin which, in one application, involves swirling a mist of liquid disinfection fluid and air around the probe's exposed surfaces followed by a drying process. Upon completion of the sterilization/disinfection process, the door of the apparatus is opened, and the probe is withdrawn. In another application, the sterilization/disinfection process involves subjecting the surface of the probe to UV or Gamma radiation.

The state of the art may benefit from more robust and versatile sterilization/disinfection processes.

### SUMMARY OF THE INVENTION

Systems and methods in accordance with embodiments of the invention act to retain disinfection/sterilization objects within a disinfection/sterilization chamber in a manner that can reduce occlusion of the disinfection with respect to the disinfecting/sterilizing media, and where there is occlusion, acts to mitigate it.

In one embodiment, there is provided a device for retaining an object in a disinfection apparatus, said device including:
at least one support member for contact engagement with one or more contact surfaces of the object,
wherein the least one support member is formed from a material that allows a disinfection source to reach the one or more contact surfaces of the object such that the one or more contact surfaces are disinfected during a disinfection process.

In one embodiment, the disinfection source is a disinfection fluid. Preferably, the disinfection fluid includes hydrogen peroxide.

In one embodiment, the at least one support member is formed from a fluid permeable material such that the disinfection fluid travels through the at least one support member to reach the one or more contact surfaces during the disinfection process.

In one embodiment, the at least one support member is formed from narrow fibres such as PET (polyester), Polypropylene and/or Nylon.

In one embodiment, the disinfection fluid travels along the surface of the at least one support member via wicking to reach the one or more contact surfaces during the disinfection process.

In one embodiment, the at least one support member is formed from a solid material such as PBT (Polybutylene terephthalate), PVDF (polyvinylidene difluoride), and/or ABS (acrylonitrile butadiene styrene).

In one embodiment, the disinfection source is UV (ultraviolet) or Gamma radiation.

In one embodiment, the at least one support member is formed from quartz glass and/or transparent/translucent plastics such as methacrylate-based polymers.

In one embodiment, the device includes a pair of support members. Preferably, the device includes a spring clamp, the clamp having the pair of support members.

In one embodiment, the at least one support member includes a net for supporting the object during the sterilization. Preferably, the net has a thickness of approximately between 0.1 and 5 mm.

In one embodiment, the at least one support member includes a basket for carrying the object during the disinfection process.

In one embodiment, the support member includes a bag adapted for carrying the object during the disinfection process. Preferably, the bag is formed from a material that substantially resists contaminant ingress such that the object remains sterile upon removal from the disinfection apparatus (e.g. Tyvek®).

In one embodiment, the device includes an elongate hanger.

In one embodiment, the at least one support member has a relatively small footprint so as to minimize the one or more contact areas of the object.

In one embodiment, the disinfection apparatus includes an enclosed chamber, and wherein the device for retaining an object is adapted to suspend the object within the chamber during the disinfection process.

In one embodiment, at least a portion of the device is impregnated with a dye that changes color when exposed to a disinfection fluid or UV or Gamma radiation for a pre-defined period.

In one embodiment, at least a portion of the device is impregnated with a dye that changes color when exposed to a pre-defined temperature for a pre-defined period.

In one embodiment, the object includes an ultrasound probe.

Reference throughout this specification to “one embodiment”, “some embodiments” or “an embodiment” means that a particular feature, structure or characteristic described in connection with the embodiment is included in at least one embodiment of the present invention. Thus, appearances of the phrases “in one embodiment”, “in some embodiments” or “in an embodiment” in various places throughout this specification are not necessarily all referring to the same embodiment but may. Furthermore, the particular features, structures or characteristics may be combined in any suitable manner, as would be apparent to one of ordinary skill in the art from this disclosure, in one or more embodiments.

### BRIEF DESCRIPTION OF THE DRAWINGS

Preferred embodiments of the invention will now be described, by way of example only, with reference to the accompanying drawings in which:

FIG. 1 is a perspective view of a device for retaining an object in a disinfection apparatus according to one embodiment of the invention.

FIG. 2 is a perspective view of a disinfection apparatus used for disinfecting medical objects;

FIG. 3 is a perspective view of a disinfection apparatus of FIG. 2 with its door open;

FIG. 4 is a perspective view of the device for retaining an object of FIG. 1, suspending an object within the chamber of the disinfection apparatus of FIGS. 2 and 3;

FIG. 5 is a perspective view of a device for retaining an object in the disinfection apparatus according to a further embodiment of the invention;

FIG. 6 is a perspective view of the device for retaining an object of FIG. 5, suspending an object within the chamber of the disinfection apparatus of FIGS. 2 and 3;

FIG. 7 is a perspective view of a device for retaining an object in the disinfection apparatus according to yet a further embodiment of the invention;

FIG. 8 is a perspective view of the device for retaining an object of FIG. 7, suspending an object within the chamber of the disinfection apparatus of FIGS. 2 and 3;

FIG. 9 is a perspective view of part of a device for retaining an object in the disinfection apparatus according to yet a further embodiment of the invention; and

FIG. 10 is a perspective view of the device for retaining an object of FIG. 9, suspending an object within the chamber of the disinfection apparatus of FIGS. 2 and 3.

### DETAILED DESCRIPTION OF THE PREFERRED EMBODIMENTS

Exemplary embodiments of the present invention will now be described in detail with reference to the accompanying drawings. In the drawings, the same elements are denoted by the same reference numerals throughout. In the following description, detailed descriptions of known functions and configurations incorporated herein have been omitted for conciseness and clarity.

It should be appreciated that the terms “disinfection” and “sterilization” are used interchangeably throughout this application as the described concepts can be applicable in either context. Similarly, that the terms “disinfect” and “sterilize” are used interchangeably throughout this application.

### Context

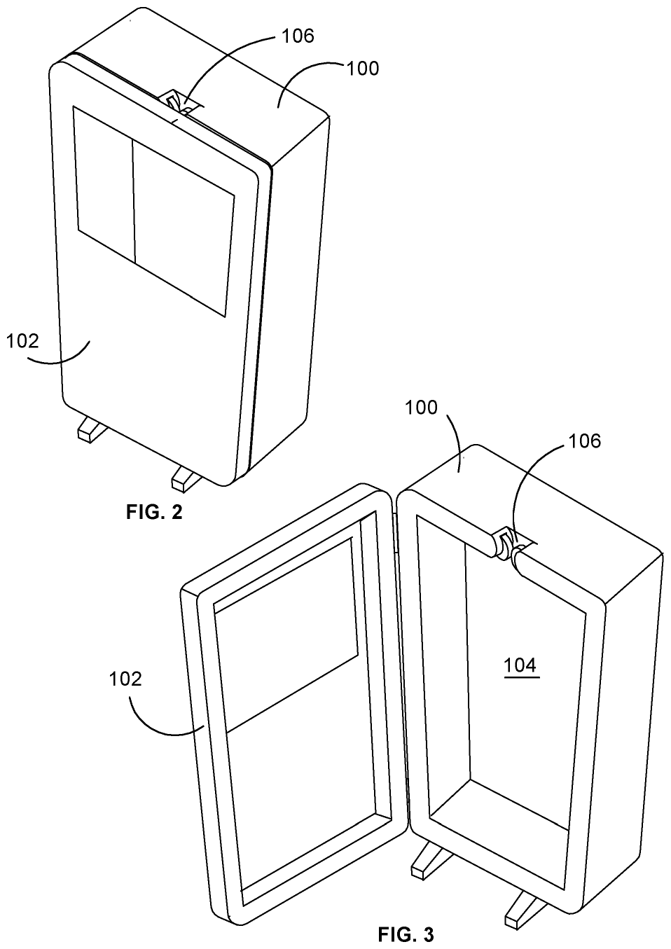

Referring initially to FIGS. 2 and 3 to outline the context for the instant application, there is shown a disinfection apparatus 100 used for disinfecting objects used in the medical industry. In particular, the Figures illustrate a disinfection apparatus 100 has been primarily designed to disinfect ultrasound probes. However, other items used in the medical industry may be disinfected using the same apparatus.

Ultrasound probes are used for a variety of intra cavity procedures including intra rectal, intra vaginal and oesophageal examination as well as surface use such as prenatal and other inspections. These probes are typically constructed integrally with the power/data cord for powering the probe and linking to a control console. Whilst the probes do not need to be completely sterile in most cases, they do need to be subjected to regular disinfection. Usually, this takes the form of at least a high-level disinfection between each use to prevent cross-contamination. In this regard, the level of contaminant removal achieved by disinfection processes are typically regulated by relevant medical standards to minimise the possibility of the cross-contamination.

With specific reference to FIG. 3 to demonstrate the use of the disinfection apparatus 100. In use, the apparatus' door 102 is first opened to expose an internal chamber 104. The probe is then placed inside the chamber 104 and secured in position. The door 102 is then closed, and the disinfection process begins by a user activation.

In one application, a disinfection media in the form of misted/vaporised disinfection fluid it is introduced into the chamber. The fluid is dispersed to swirl around the ultrasound probe during a disinfection process. This fluid dispersion is followed and introduction of a flow of air to dry the probe's outer surfaces. At the completion of this process, the outer surfaces of the probe are disinfected.

The misted disinfection fluid may include a quantity of controlled ultrafine hydrogen peroxide (H2O2), which enters the chamber through side ports and gently swirls around to cover the entire surface of the probe. The mist particles can penetrate exposed areas of the probe including shadowed areas formed by crevices, grooves and imperfections on the probe surface. As part of the disinfection process, the mist flow is stopped, and a flow of air is introduced to the inside of the chamber so that the exposed surfaces of the probe can dry to a sufficient level. In this regard, it should be understood, that the drying of the probe surfaces can be an important aspect of the disinfection process when using a misted disinfection fluid so that the disinfection fluid can be substantially removed from the probe surfaces at the completion.

In another application, the disinfection media is in the form of UV (ultraviolet) or Gamma radiation, emitted from internally mounted lamp(s) or other source(s), to reach the external surfaces of the object undergoing disinfection. UV or Gamma radiation has a strong bactericidal effect. It can be absorbed by the DNA of any microorganism that is present, to destroy its structure and inactivate any living cells. Microorganisms such as viruses, bacteria, yeasts and fungi may be rendered harmless within seconds by UV radiation.

In either of the two above-mentioned applications, the probe is normally secured within the chamber 104 of the disinfection apparatus 100 by way of a clamp 106 disposed at the top of the chamber. This clamp 106 is intended to engage with the probe's power/data cord thereby allowing it to be suspended within the chamber 104 during the disinfection process. However, a problem now exists whereby ultrasound probes, and indeed other medical objects requiring disinfection, have been developed with internal power sources and wireless communication devices such that they do not require an external cord. It should be appreciated that in the context of suspending the probe within chamber 104, the absence of a cord is problematic in that simply resting the wireless probe within the chamber can inhibit the disinfection media reaching those areas that are in contact with the chamber wall. Similarly, suspending the probe within the chamber using a conventional bracket, will result in the areas of the probe in contact with the bracket not being sufficiently disinfected due to the bracket effectively blocking the disinfection media. In other words, such supportive structures may hinder the disinfection media from reaching respective occluded surfaces. Under these circumstances, the level of contaminant removal may be insufficient to meet regulated medical standards.

### Development

With this problem in mind, systems and methods in accordance with embodiments of the invention act to retain a target of disinfection within a disinfection chamber while reducing instances of occlusion and mitigating detrimental impacts of the occlusion—where it does occur—that may otherwise result from corresponding supportive structures. By contrast, conventional solutions for addressing disinfection of mated surfaces have included UV-transparent trays, which are bulky and can more directly interfere with the disinfecting media (the trays of course may not be applicable in a chemical disinfectant fluid context in any case). Moreover, in many embodiments of the invention, the retention mechanism is flexible and can thereby accommodate a variety of form-factors. For example, in many embodiments of the invention, a netting is implemented for retaining the disinfection target within a disinfection chamber, and the netting may be constructed so as to be ‘effectively transparent’ to the disinfecting media. The combination of the fact that netting is inherently porous and is made of material that is effectively transparent to disinfecting media can allow the disinfecting media to efficiently interact with the surface of the target object.

For example, in several embodiments of the invention, a netting is implemented that is permeable to a disinfection fluid, e.g. aerosolized H2O2. Thus, for instance, such a netting can be used with a disinfection apparatus that utilizes H2O2 as the disinfection media. Although placing a disinfection target within the netting will necessarily create mating surfaces, aerosolized H2O2 may still reach the respective mating surfaces of the target since the netting is permeable to it. In some embodiments, the netting is made of a material that is characterized by good wicking properties that can facilitate the disinfection chemistry reaching the otherwise occluded surface of the disinfection target object. In essence, the netting can be said to be ‘effectively permeable.’ Suitable materials for creating a netting that is so permeable include polyester (e.g. polyethylene terephthalate [“PET”]), polypropylene, and/or nylon. Of course, it should be appreciated that any suitable material can be implemented that can allow a disinfection fluid to reach a mated surface may be implemented in accordance with embodiments of the invention.

In many embodiments, a porous basket that is effectively transparent to disinfection fluids is implemented. As with the above-described netting, a basket can be advantageous insofar as it can accommodate a variety form factors. Similarly, as before, the apertures present in a basket can more directly allow a disinfection fluid to interact with the target's surface. A basket may be further advantageous insofar it can provide the desired structural rigidity. As before, the basket can be made of any suitable material in accordance with embodiments of the invention. For example, the basket can be made of materials such as: polybutylene terephthalate, polyvinylidene fluoride, and/or acrylonitrile butadiene. Without being bound by theory, it is believed that such materials are characterized by good wicking properties, and would facilitate capillary action that would allow disinfection fluids to navigate to a contacted portion of a target object's surface and disinfect those occluded areas.

In some embodiments, supportive structures are characterized by a relatively small footprint, yet can still suspend a disinfection target within a disinfection chamber can be implemented. Advantageously, having a small footprint can minimise contact areas that would otherwise be occluded during the disinfection process. Examples of supportive structures with relatively small footprints may include clamping mechanisms. The mating part(s) of the supportive structures can be made to be effectively transparent to disinfecting media. For example, such parts can implement materials that facilitate wicking at the mated surfaces, or travel along the structure surface, such that fluid disinfectant can reach the mated surface, thereby enabling disinfection. Examples of such materials include: polybutylene terephthalate (PBT), polyvinylidene difluoride (PVDF), and/or acrylonitrile butadiene styrene (ABS). Of course, it should be appreciated that any suitable material can be implemented in accordance with embodiments of the invention.

As mentioned previously, many disinfection apparatuses utilize electromagnetic radiation to disinfect target objects. Accordingly, many embodiments act to retain a target object of disinfection within an electromagnetic radiation-based disinfection chamber while (a) reducing occlusion (e.g. relative to conventional solutions), and (b) mitigating detrimental impacts of the occlusion that may otherwise result from corresponding supportive structures. Thus, for instance, the above-described nettings and baskets may be made from material that is transparent to UV-radiation and/or gamma radiation. Similarly, the above-described clamping structures can be made from materials that are transparent to UV-radiation and/or gamma radiation. For example, suitable materials may include: quartz glass and/or transparent/translucent methacrylate-based polymers. Of course, it should be appreciated that any suitable material may be used in accordance with embodiments of the invention.

As can be appreciated, the above-described structures can provide efficient mechanisms for retaining disinfection targets within a disinfection chamber while improving surface area exposure so that disinfection media can more directly interact with the target object's surface. Moreover, the structures can enable the disinfection media to reach occluded surfaces where there is occlusion. In this way, the retention mechanisms can allow the disinfection media to efficiently interact with the object's surface, while simultaneously providing sufficient structural rigidity. Moreover, it should be noted that many of the disclosed embodiments herein can be made to accommodate chemical disinfection. As will be illuminated by the forthcoming examples, many of the disclosed embodiments can be further advantageous insofar as they can be made to couple with the heritage disinfection systems. For example, many heritage disinfection systems utilize clamping mechanisms from which to suspend ultrasound probes from via their embedded cables. Certain embodiments disclosed herein can similarly be made to interface with such mechanisms, and therefore enable heritage systems to disinfect wireless medical instruments.

As can be appreciated, the above-described concepts can be implemented in any of a variety of ways in accordance with embodiments of the invention. Specific examples will now be presented.

### First Example

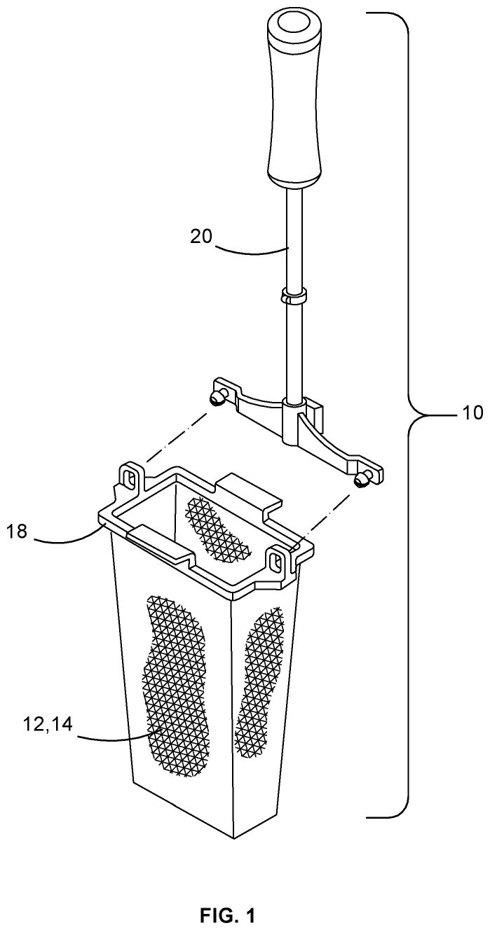

With reference now to FIG. 1, there is provided a device 10 for retaining an object in the disinfection apparatus 100 according to one embodiment of the invention. In the present example, it is proposed that the object is a wireless ultrasound probe 16, but the object could be any other object that requires disinfection. Further, in this embodiment, it should be appreciated that the proposed disinfection media is a disinfection fluid.

As mentioned earlier, when using a disinfection fluid, the disinfection process may involve misting or vaporising the disinfection fluid within the chamber of the disinfection apparatus 100 to disperse and swirl the fluid around the probe for a given period. In this regard, to retain the probe 16 in the apparatus 100, the device 10 includes a support member 12 for contact engagement with one or more contact surfaces of the probe during the disinfection apparatus. According to the present embodiment, the support member is formed, at least partially in the contact areas with the probe, from a fluid permeable material that allows the disinfection fluid to reach the contacted or occluded surfaces. For example, the material may be directly permeable to the fluid and/or it may be ‘effectively permeable’ insofar as the material is characterized by good wicking properties that allow the disinfectant media to reach the contact surface. In this way, the misted/aerosolized disinfection fluid can reach the mated surface and disinfect it. In the illustrated embodiment, the at least one support member 12 includes a net 14 which is used to contain and suspend the object during the disinfection process.

Having the fluid permeable material in contact with the probe during disinfection can allow the disinfection mist/aerosol to permeate through the contact points of the support member to the surfaces of the ultrasound probe 16 during the disinfection process. By having the net formed from a fluid permeable material, the disinfection fluid can also more easily evaporate from the surfaces of the probe during the drying stage of the sterilization process, i.e., when the swirling of disinfection fluid mist is completed.

The device 10 further includes a retaining ring 18, which is removably attached to the net 14. An elongate hanger 20 is further provided for removable engagement with the ring 18 thereby supporting the net 14. Once the hanger 20 is engaged with ring 18, the probe, or other object, may be placed within net.

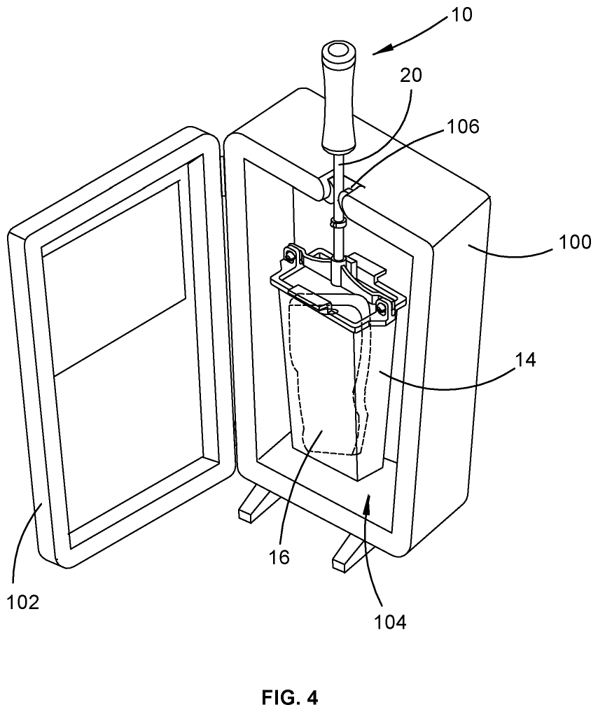

As best shown in FIG. 4, the hanger 20 can then be placed in the clamp 106 at the top of chamber 104 so that the net and probe 16 is suspended within the chamber but not in contact with the chamber walls. The door 102 of the disinfection apparatus 100 can then be closed and the disinfection process can then begin by user activation.

As should be understood, hanger 20 and the retaining ring 18 do not need to be formed from a fluid permeable material and, in some cases, the hanger may be in the form of a flexible cable without departing from the scope of the invention.

Advantageously, due to the fluid permeability of the net 14, the disinfection mist can penetrate the net to contact all external surfaces of the probe 16 during the disinfection process including the portions in contact with the net material. Similarly, once the disinfection mist is completed and the drying phase begins, the fluid permeability of the net can allow the disinfection fluid on surfaces of the probe to evaporate to a sufficient level.

The net 14 can be formed from a fabric material having narrow fibres of PET (polyester), polypropylene and/or nylon. These materials can provide sufficient fluid permeability to achieve the required level of fluid transference to provide sufficient disinfection of the probe surfaces during the given disinfection cycle.

In the present embodiment, the net 14 may be formed from a mesh of polyester polymer. The thickness of the net material may be a consideration in this context of the disinfection function. For example, if the net material is too thick, the level of fluid permeability of disinfection agent during the disinfection cycle may be insufficient to coat the external surfaces of the probe. On the other hand, if the net material is too thin, the net may be structurally insufficient to both retain the probe and/or other objects suspended during the disinfection cycle and may risk collapsing during the cycle. Furthermore, it should be appreciated that it can be advantageous that the net material be sufficiently structurally and materially resistant to mitigate deterioration during multiple uses and while it is being stored for extended periods prior to use. In this regard, the inventor has found that when using polyester polymer material for the net, a thickness of between 0.1-5 mm, and preferably 0.2 mm, can provide a sufficient level of fluid transferral and will be sufficiently structurally resilient.

As alluded to previously, one advantage of a netting is that surfaces of target object to be disinfected may be directly exposed to the disinfecting fluid via the inherent pores of the netting. In many instances, significant portions of the target object may be so exposed. This can result in more efficient disinfection. Nettings being characterized by any suitable aperture size may be implemented in accordance with embodiments of the invention. For example, aperture sizes between approximately 0.1 mm and approximately 10 mm may be implemented.

### Second Example

In a variation to the first example, instead of the above-mentioned net 14, the at least one support member may be in the form of a sterile bag formed of a fluid permeable material. In a similar way to the earlier embodiment, the bag containing the object to be sterilised can be suspended within the sterilization chamber 104 using hanger 20 or similar means, during the sterilization/disinfection process. Further, the fluid permeability of the bag material can allow a mist of disinfection fluid to contact the exposed surfaces of the object contained within the bag during the sterilization/disinfection process. At the same time, the fluid permeable material of the bag can also allow the disinfection fluid to evaporate from the exposed surfaces of the contained object during the drying cycle. However, the bag differs from the net 14 of the earlier embodiment in that it is formed from a material that can inhibit the ingress of contaminants after sterilization/disinfection. In particular, the bag can inhibit the ingress of contaminants into the sealed bag once it is removed from the sterilization/disinfection apparatus. Therefore, once the sterilization/disinfection process is completed, the bag can be removed from the chamber and can remain sealed until the object is required to be used. In this way, handling of the object can become simpler and can remain sterile for a longer period. Suitable materials for forming the bag include Tyvek®.

### Third Example

A variation of the net 14 and hanger 20 of the previous embodiment is shown in FIG. 5 according to a further embodiment of the invention. As shown, hanger 20 is now in the form of elongate hanger 22 having a hook portion 24 at one end and ring 26 at the other end. In this embodiment, the at least one support member 12 is now in the form of net 28 having support aperture 30. The support aperture is engageable with the hook portion 24 of the hanger 22, so that the net may suspended in the chamber 104. The net 28 may be formed from the same fluid permeable material as net 14 to allow flow of the disinfection fluid to flow to the surface of the object, as well as allow the disinfection fluid to dry during the drying process.

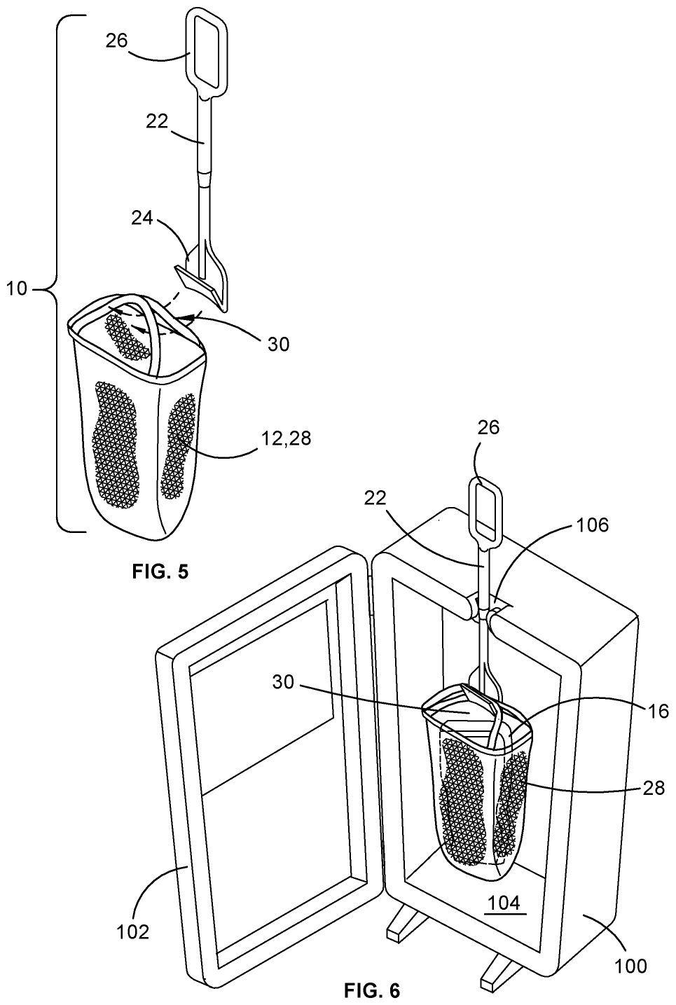

As shown in FIG. 6, in this embodiment, the hook portion 24 engages with support aperture 30 of the net 28 to support the net 28. The hanger 22 can then be placed in clamp 106 in a similar way to the first embodiment so as to retain the object within disinfection apparatus 100. However, in this embodiment, there is no requirement for a separate retaining ring 18. Moreover, it should be noted that elongate hanger 22 can be used with other variations of the support member as discussed further below.

### Fourth Example

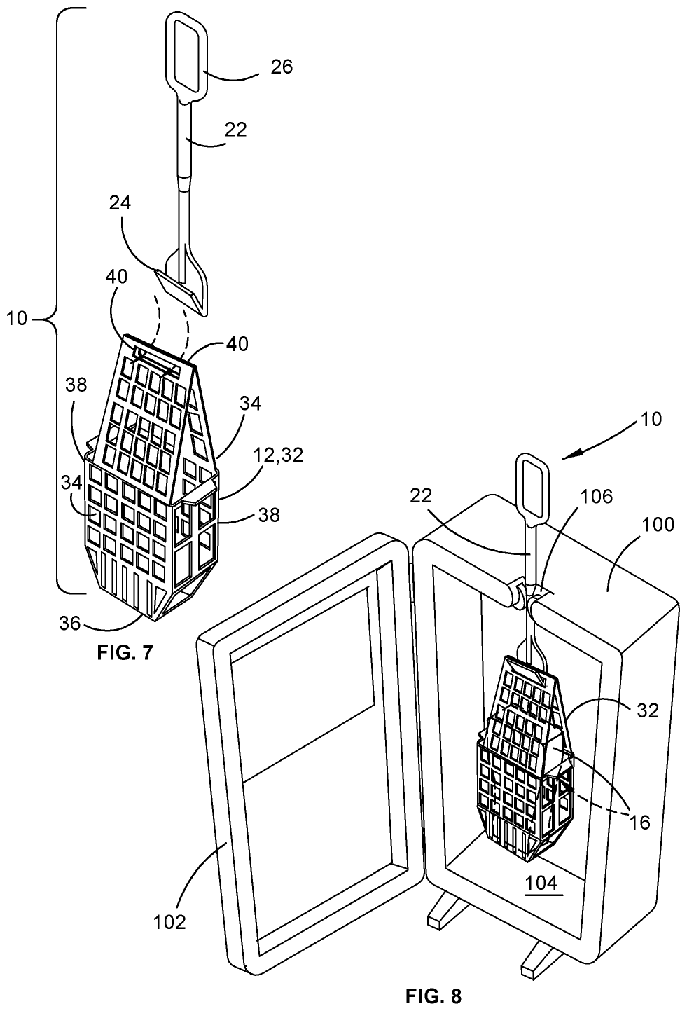

With reference now to FIG. 7, the device 10 for retaining an object in a disinfection apparatus is shown according to a further embodiment of the present invention. In this embodiment, the support member 12 includes a basket 32, having opposed front and back sides 34, a bottom portion 36 and a pair of opposed side walls 38.

The front and back sides of the basket include support apertures 40, which are engageable with the hook portion 24 of the elongate hanger 22 as mentioned with reference to the previous embodiment.

As shown in FIG. 8, as with the previous embodiments, the object to be disinfected is placed in the basket 32 and hung in the chamber 104 of the disinfection apparatus 100. Under these circumstances, the hanger 22 engages with the clamp at the top of the chamber 106 to retain the object in the chamber.

The basket 32 according to this embodiment can also be formed from a fluid permeable material so that once the disinfection fluid is dispersed within the chamber, the disinfection fluid can pass through the basket to reach the contact surfaces of the object. In one possible variation, it is proposed that the basket 32 be formed of natural polypropylene which can have the required fluid permeability characteristics. Of course, it can be appreciated that any suitable material can be implemented in accordance with embodiments of the invention. For example, polyethylene terephthalate (“PET”) and/or nylon may be utilized. Advantageously, due to the larger apertures provided, the basket 32 can provide less resistance for fluid to contact the surfaces of the object to be disinfected than the nets of the first and second examples. Suitable aperture sizes for the basket may include sizes greater than 0.1 mm. As can be appreciated, the basket can be characterized by any suitable thickness in accordance with embodiments of the invention. For example, the basket can be implemented with a thickness of between approximately 0.1 mm and approximately 5 mm. Note that while a particular geometry has been illustrated and described, it should of course be appreciated that baskets of any suitable geometry can be implemented in accordance with embodiments of the invention. For example, baskets can be cylindrical or have polygonal base in accordance with embodiments of the invention.

### Fifth Example

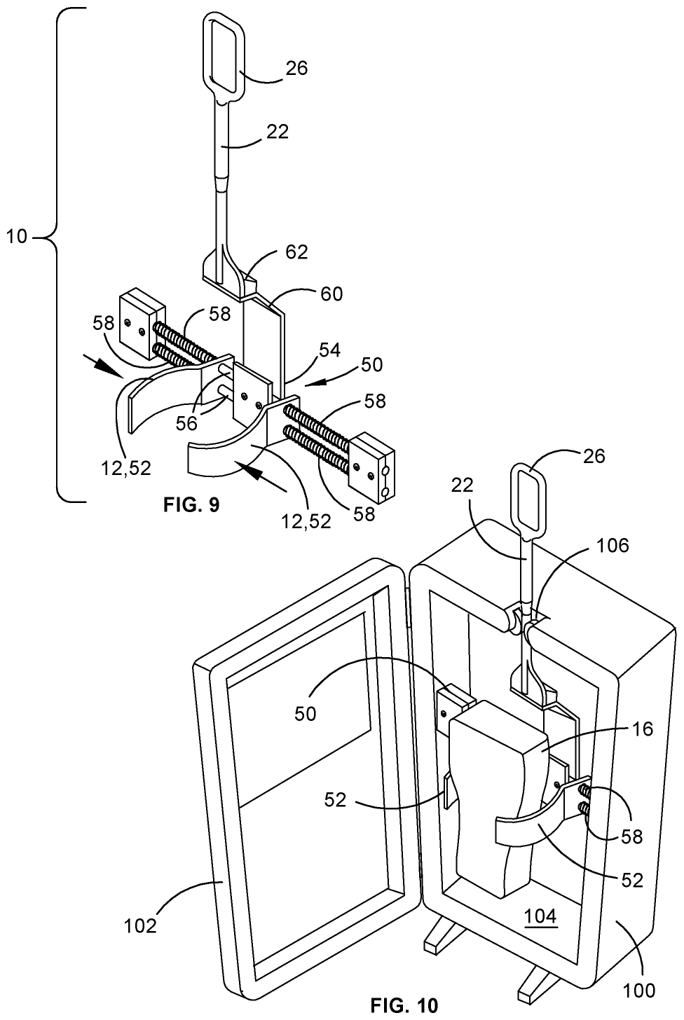

With reference now to FIG. 9, the device 10 for retaining an object in a disinfection apparatus is shown according to a further embodiment of the present invention. In this embodiment, the device 10 includes a spring clamp 50.

Spring clamp 50 includes a pair of support members 12 in the form of a pair of opposed fingers 52. Each finger is engaged with tracks 56 mounted to a base 54. By this engagement, the fingers are adapted to slidably move between a closed configuration and an open configuration. Further, by provision of springs 58, the fingers 52 are biased into the closed configuration so as to retain and a hold an object therebetween. A link arm 60 is provided and attached to the base 54 at one end using screw or other coupling mechanisms, and engaged with the elongate hanger 22, at the other end by way of screw mount 62.

As with previous embodiments, the fingers 52, which are proposed to be in contact with the object to be disinfected, can be formed from a material that allows the disinfection fluid to reach the contact surfaces, e.g. via wicking. Such materials include PBT, PVDF, and/or ABS, which can have good wicking properties. As with the previous embodiment, only the fingers 52 are required to be formed from such materials. The remaining components can be formed of any material suitable for purpose.

As best shown in FIG. 10, in use, a separation force is applied to separate the fingers 52 against the biasing force of the springs 58. Once separated, the object is placed between the fingers 52 and retained by the biasing force. The assembly of the fingers 52, base 54, link arm 60, and elongate hanger 22, is then placed into the chamber 104 to be held by the clamp 106 at the top of the chamber. The door 102 of the disinfection apparatus 100 can then be closed and the disinfection process can then begin by user activation. During this process, disinfection fluid is again misted and dispersed around the object. The effective permeability of the fingers can allow disinfectant to reach contact surfaces.

In some embodiments, supportive structures characterized by a relatively small footprint, yet can still suspend a disinfection target object within a disinfection chamber can be implemented. Examples of supportive structures with relatively small footprints may include the spring clamp discussed above. The mating part(s) of said supportive structures can be made to be effectively transparent to disinfecting media. For example, such parts can implement materials that facilitate wicking at the mated surfaces such that chemical disinfectant can reach the mated surface, thereby enabling disinfection. Examples of such materials include: polybutylene terephthalate (PBT), polyvinylidene difluoride (PVDF), and/or acrylonitrile butadiene styrene (ABS). Of course, it should be appreciated that any suitable material can be implemented in accordance with embodiments of the invention.

As can be appreciated, the disclosed spring clamp may offer better structural support relative to for example merely hanging the disinfection target object. Additionally, by leveraging the clamping force, the contact areas can be effectively reduced.

As can be appreciated, the above-described concepts can be implemented in a variety of ways in accordance with embodiments of the invention. Accordingly, the above-described examples are meant to be just that—examples of how the concepts may be implemented, and not an exhaustive list of embodiments of the invention. For example, a single device may include different forms, or multiple support members, so that multiple objects can be retained in the apparatus at the same time.

### UV/Gamma Radiation

As mentioned previously, some disinfecting apparatus utilize electromagnetic radiation to disinfect target objects. For example, UV-radiation and gamma radiation may be effective to disinfect target objects. Accordingly, the inventive concepts of the instant application may be extended in this context as well. For example, retaining structures can be made of materials that are transparent to such radiation. In this regard, each of the retaining structures of the above-described examples can be constructed from materials transparent to respective electromagnetic radiation. Thus, for instance:
In the first example, the net 14 may be made of material that is effectively transparent to UV-radiation and/or gamma radiation
In the second example, the bag may be formed of a material that is effectively transparent to UV-radiation and/or gamma radiation.
In the fourth example, the basket 32 may be made of a material that is effectively transparent to UV-radiation and/or gamma radiation.
In the fifth example, the fingers 52 can be made of a material that is effectively transparent to UV-radiation and/or Gamma radiation.

Thus, accessories for use within disinfection chambers that are based on the application of electromagnetic radiation can be achieved. Suitable materials that are transparent to electromagnetic radiation can include quartz glass and transparent/translucent plastics such as methacrylate-based polymers.

As can be appreciated, the above-described structures can be advantageous relative to conventional UV-transparent trays, at least insofar as the above-described structures offer reduced mating surfaces. The more direct interaction between the electromagnetic radiation and the target surface can yield more efficient disinfection.

### Other Aspects

The above-described examples can be augmented and enhanced in various ways in accordance with embodiments of the invention. For example, in some embodiments, the materials selected can allow for a decrease in disinfection performance at the points of contact between the support member and the object undergoing sterilisation and this can be addressed by operating a specific cycle of the sterilisation apparatus, where the disinfection potency is increased to mitigated the projected performance degradation.

In several embodiments, a support member is a limited use item. Under these circumstances, at least a portion of the support member may be impregnated with a dye that reacts to the disinfection media so that the support member will change color at the completion of a given number of disinfection processes. In this way, a visual indication is provided to the user to easily determine if the device is suitable for use.

In a number of embodiments, it is proposed that at least a portion of the effectively transparent material used for the support member, is impregnated with a dye that reacts to a predefined temperature created during the disinfection process, so that the support member will change color at the completion of a given number of disinfection processes. The predefined temperature may be any as selected, and in one application, may be a temperature of more than 50 degrees Celsius. In this way, a visual indication is provided to the user to easily determine if the device is suitable for use.

In several embodiments, the support member allows for attachment of a unique identifier, to enable traceability of the supporting member to the particular disinfection cycle. The unique identifier may be in the form of a digital solution such as a RFID tag or barcode/QR code. Alternatively, the unique identifier may be an analogue solution such as a serial number. In another embodiment, the supporting member shall have an embedded unique identifier, to enable traceability of the supporting member to the particular disinfection cycle.

In one embodiment, the support member 12 is provided to the user in a sterile packaging provided with a “use by” date that indicates the date where the seal of the packaging is no longer assured.

Advantageously, the present invention provides a device to retain objects in a disinfection apparatus so that all exposed surfaces of the objects are exposed to a disinfection media during the disinfection process.

It should be appreciated that in the above description of exemplary embodiments of the invention, various features of the invention are sometimes grouped together in a single embodiment, Figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of this invention.

In the description provided herein, numerous specific details are set forth. However, it is understood that embodiments of the invention may be practiced without these specific details. In other instances, well-known methods, structures and techniques have not been shown in detail in order not to obscure an understanding of this description.

While there has been described what are believed to be the preferred embodiments of the invention, those skilled in the art will recognize that other and further modifications may be made thereto without departing from the spirit of the invention, and it is intended to claim all such changes and modifications as falling within the scope of the invention. For example, any formulas given above are merely representative of procedures that may be used. Functionality may be added or deleted from the block diagrams and operations may be interchanged among functional blocks. Steps may be added or deleted to methods described within the scope of the present invention.

1. A device for retaining an object in a disinfection apparatus, said device including:
at least one support member for contact engagement with one or more contact surfaces of said object,
wherein said at least one support member is formed from a material that allows a disinfection media to reach said one or more contact surfaces of said object such that said one or more contact surfaces are disinfected during a disinfection process.

2. A device for retaining an object in a disinfection apparatus according to claim 1, wherein said disinfection source is a disinfection fluid.

3. A device for retaining an object in a disinfection apparatus according to claim 2, wherein said disinfection fluid includes hydrogen peroxide.

4. A device for retaining an object in a disinfection apparatus according to claim 2 or claim 3, wherein said at least one support member is formed from a fluid permeable material such that the disinfection fluid is able to travel through said at least one support member to reach the one or more contact surfaces during said disinfection process.

5. A device for retaining an object in a disinfection apparatus according to any one of the preceding claims, wherein said at least one support member is formed from narrow fibres such as PET (polyester), Polypropylene and/or Nylon.

6. A device for retaining an object in a disinfection apparatus according to any one of claims 2 to 5, wherein said disinfection fluid travels along the surface of said at least one support member to reach said one or more contact surfaces during said disinfection process.

7. A device for retaining an object in a disinfection apparatus according to any one of the preceding claims, wherein said at least one support member is formed from a solid material such as PBT (Polybutylene terephthalate), PVDF (polyvinylidene difluoride), and/or ABS (acrylonitrile butadiene styrene).

8. A device for retaining an object in a disinfection apparatus according to claim 1, wherein said disinfection media is UV (ultraviolet) or Gamma radiation.

9. A device for retaining an object in a disinfection apparatus according to any one of the preceding claims, wherein said at least one support member is formed from quartz glass and/or transparent/translucent plastics such as methacrylate-based polymers.

10. A device for retaining an object in a disinfection apparatus according to any one of the preceding claims, including a pair of support members.

11. A device for retaining an object in a disinfection apparatus according to claim 6 including a spring clamp, said clamp having said pair of support members.

12. A device for retaining an object in a disinfection apparatus according to any one of the preceding claims, wherein said at least one support member includes a net for supporting said object during said disinfection process.

13. A device for retaining an object in a disinfection apparatus according to claim 10, wherein said net has a thickness of approximately between 0.1 and 5 mm.

14. A device for retaining an object in a disinfection apparatus according to any one of the preceding claims, wherein said at least one support member includes a basket for carrying the object during the disinfection process.

15. A device for retaining an object in a disinfection apparatus according to any one of the preceding claims, wherein said support member includes a bag adapted to support said object during said disinfection.

16. A device for retaining an object in a disinfection apparatus according to claim 13, wherein said bag is formed from a material that substantially resists contaminant ingress such that said object remains sterile upon removal from said disinfection apparatus.

17. A device for retaining an object in a disinfection apparatus according to any one of the preceding claims, including an elongate hanger.

18. A device for retaining an object in a disinfection apparatus according to claim 10 or claim 11, wherein said at least one support member has a relatively small footprint so as to minimize said one or more contact areas of the object.

19. A device for retaining an object in a disinfection apparatus according to any one of the preceding claims, wherein said disinfection apparatus includes an enclosed chamber, and wherein said device for retaining an object is adapted to suspend said object within said chamber during said disinfection process.

20. A device for retaining an object in a disinfection apparatus according to any one of the preceding claims, wherein at least a portion of said device is impregnated with a dye that changes color when exposed to a disinfection fluid or UV or Gamma radiation for a pre-defined period.

21. A device for retaining an object in a disinfection apparatus according to any one of the preceding claims, wherein at least a portion of said device is impregnated with a dye that changes color when exposed to a pre-defined temperature for a pre-defined period.

22. A device for retaining an object in a disinfection apparatus according to any one of the preceding claims, wherein said object includes an ultrasound probe.
